(19) United States
(12) Patent Application Publication
(10) Pub. No.: US 2015/0265417 A1
(43) Pub. Date: Sep. 24, 2015

(54) SUPPORT DEVICE AND METHOD

(71) Applicant: Flexmedex, LLC, Quakertown, PA (US)

(72) Inventors: E. Skott GREENHALGH, Gladwyne, PA (US); John-Paul ROMANO, Chalfont, PA (US)

(73) Assignee: FLEXMEDEX, LLC, Quakertown, PA (US)

(21) Appl. No.: 14/730,147

(22) Filed: Jun. 3, 2015

Related U.S. Application Data
(63) Continuation of application No. 13/573,542, filed on Sep. 21, 2012, now abandoned.
(60) Provisional application No. 61/537,386, filed on Sep. 21, 2011.

(51) Int. Cl. A61F 2/44; A61B 17/84; A61B 17/70; A61F 2/46

(52) U.S. Cl. CPC A61F 2/4455; A61F 2/4611; A61B 17/844; A61B 17/7055; A61F 2002/30018

(57) ABSTRACT

Devices and methods for orthopedic support are disclosed. The device can have a first rigid section hingedly attached to a second rigid section. A tunnel through the bone near the implantation target site can be created. The device can be inserted into and pass through and out of the tunnel to the target site.

### CROSS-REFERENCE TO RELATED APPLICATION

The present application is a continuation of U.S. patent application Ser. No. 13/573,542 filed Sep. 21, 2012 which claims the benefit of U.S. Provisional Application No. 61/537,386 filed Sep. 21, 2011, each of which are herein incorporated by reference in its entirety.

### BACKGROUND OF THE INVENTION

1. Field of the Invention

A device, such as a flexible spinal fusion cage, which can articulate or bend in such a way that it will be able to be implanted through bone (i.e., in a trans-osseous path, through bone, such as the Ilium and/or sacrum joint approach into L5-S1 is disclosed.

2. Description of the Related Art

Typical lateral approach fusion implants are not able to implant fusion cages in the lower lumbar region for at least two reasons. First, boney obstacles can impair access. FIGS. 1a and 1b illustrate the challenge of gaining lateral access to L4-L5 and L5-S1. Note the position of the Ilium relative to the direct lateral access pathway. The Ilium obstructs the target site for typical approaches to the respective disc spaces.

Some doctors create large windows, shown in phantom lines in FIG. 2, through the Ilium to gain direct line of site access. This is a highly invasive approach and requires significant surgical skill. Because of the inflexibility of the typical implants, the windows must be large enough to fit the entire implant cross section.

Second, the approach angle of a tissue retractor relative to the location of the fusion site is an issue. The tissue retractor used in lateral fusion surgery provides line-of-site access to the disk space that is the target site for the fusion cage insertion. The retractor holds tissue out of the way. They also create a working channel to pass tools through, they protect neural tissue, and they anchor to the superior and inferior vertebral bodies relative to the disk space requiring fusion. Anything inferior to the iliac crest's lateral plane 10 is very hard, if not impossible, to reach with a direct lateral approach due to the physical obstruction created by the position and shape of the Ilium. Even if the retractors are tilted as shown by an L5-S1 delivery path 11a or L4-L5 delivery path 11b and respectively corresponding L5-S1 approach angle 12a and an L4-L5 approach angle 12b, the ability to insert an implant that is the length of the end plates of the L4 and L5 vertebral bodies would be very difficult.

Furthermore, with the retractor positioned in the plane and direction as shown by the delivery path 11a, the approach angle 12a formed between the delivery path 11a and the adjacent vertebral bodies' end plates would make inserting a monolithic fusion cage virtually impossible without severely damaging the surrounding vertebrae and/or the ilium. FIG. 5 illustrates that a typical lateral fusion cage 14 has a device width 16 about equal to the width of the vertebral end plates and has a device height 18 about equal to the height of the disk. The stiff and rigid monolithic implant can be difficult if not impossible to turn around the corner 20 at the lateral and/or anterior edge of the L5-S1 intervertebral space.

Typical treatments for L5-S1 include anterior approaches include insertion through the front of the abdomen, transforaminal lumbar interbody fusion (TLIF), and posterior lumbar interbody fusion (PLIF). Anterior and TLIF approaches are the most used. Both approaches are highly invasive and destructive to surrounding tissue.

Accordingly devices and methods for lumbar stabilization that are less destructive to surrounding tissues are desired. For example, a device and method of inserting a strong support device through a minimal channel in the ilium and/or ala that circumvent nerves and blood vessels is desired.

### SUMMARY OF THE INVENTION

Support or fixation devices and methods for access, controlling (e.g., steering) implants, and modifying implants are disclosed.

The device can be an implantable fixation device, such as a flexible fusion cage. The device can articulate and/or bend so the device can be delivered through a channel in one or more bones and into the L5-S1 intervertebral space, as shown in FIG. 6. The implant can articulate and be steered. For example, the implant can have hinges and/or be flexible.

A stand-alone fusion system and method is disclosed that can include deploying the support device with the transosseous delivery approach and optionally deploying screws and using targeting fixtures.

A biological implant support device for providing orthopedic support is disclosed. The device can have a first rigid section, and a second rigid section. The first rigid section can be at a first terminal longitudinal end of the device. The second rigid section can be rotatably attached to the first rigid section at a longitudinal end of the first rigid section away from the first terminal longitudinal end of the device. The second rigid section can have threads.

The device can have an axle rotatably attaching the first rigid section to the second rigid section.

A system for providing orthopedic support is disclosed that can have the the support device described herein and a first screw attached to and extending away from device. The system can have a plug abutting the device.

A method for inserting a support device to a target site in a spine adjacent to a first vertebra is also disclosed. The method can include creating a tunnel or channel through the ala of the sacrum. The method can include inserting a first rigid section of the device through the channel and into the target site. The method can include inserting a second rigid section of the device into the tunnel. The method can include rotating the second rigid section of the implant with respect to the first rigid section. The first rigid section can be hingedly attached to the second rigid section. The method can include fixing the second rigid section of the implant in the tunnel.

Creating the channel can include drilling with a flexible drill. The channel can have a cylindrical cross-section. The non-vertebral bone can include the pelvis, the ilium, the sacrum, or combinations thereof.

### SUMMARY OF THE DRAWINGS

FIGS. 1a and 1b are direct anterior and anterior perspective views of a variation of the lower lumbar spine.

FIG. 2 is a lateral view of the lower lumbar spine with L5 and S1 shown in phantom views behind the Ilium.

FIG. 3 is the view of the spine shown in FIG. 1 with L5-S1 and L4-L5 implant device delivery paths and approach angles.

FIG. 4 is the view of the spine shown in FIG. 2 with the L5-S1 implant device delivery path and approach angle.

FIG. 5 is the view of the spine shown in FIG. 3 with L5-S1 implant device shown being inserted along a delivery path at an approach angle.

FIGS. 6 and 7 are anterior and lateral views, respectively, of a variation of a delivery path for an implant device through the sacrum.

FIGS. 8a and 8b illustrate variations of and a delivery method for the implant support device.

FIGS. 9a through 9c illustrate a variation of the implant support device and variations of delivery methods.

FIG. 10 illustrates a variation of a plug and a method for delivering the plug into the delivery channel through bone.

FIGS. 11a and 11b are superior views of the sacrum and variations of methods for deploying multiple implant devices.

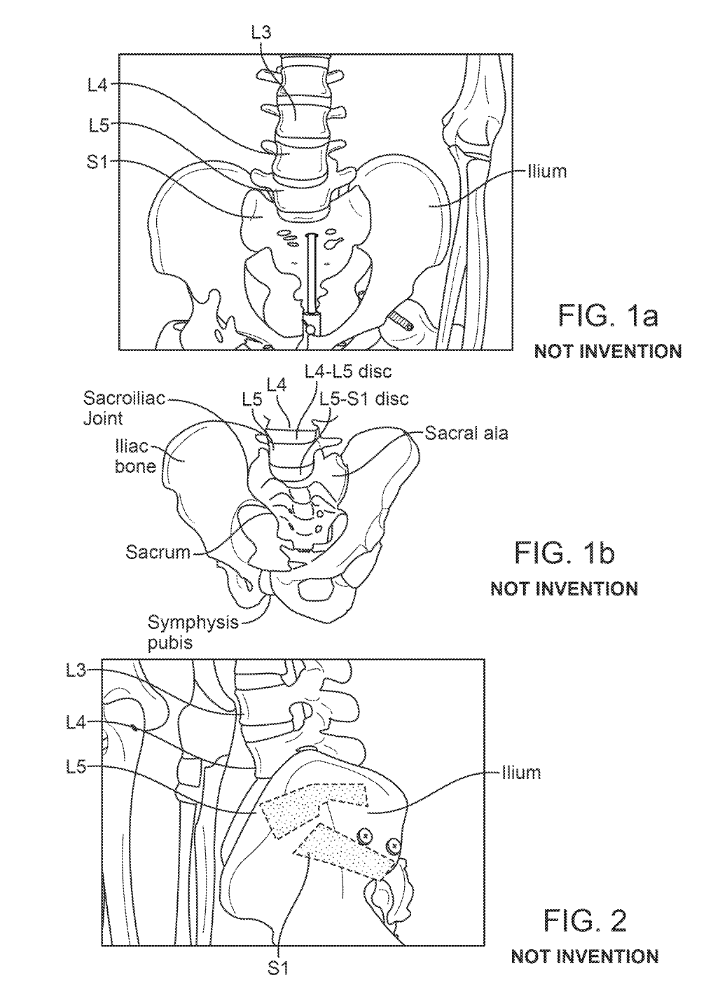

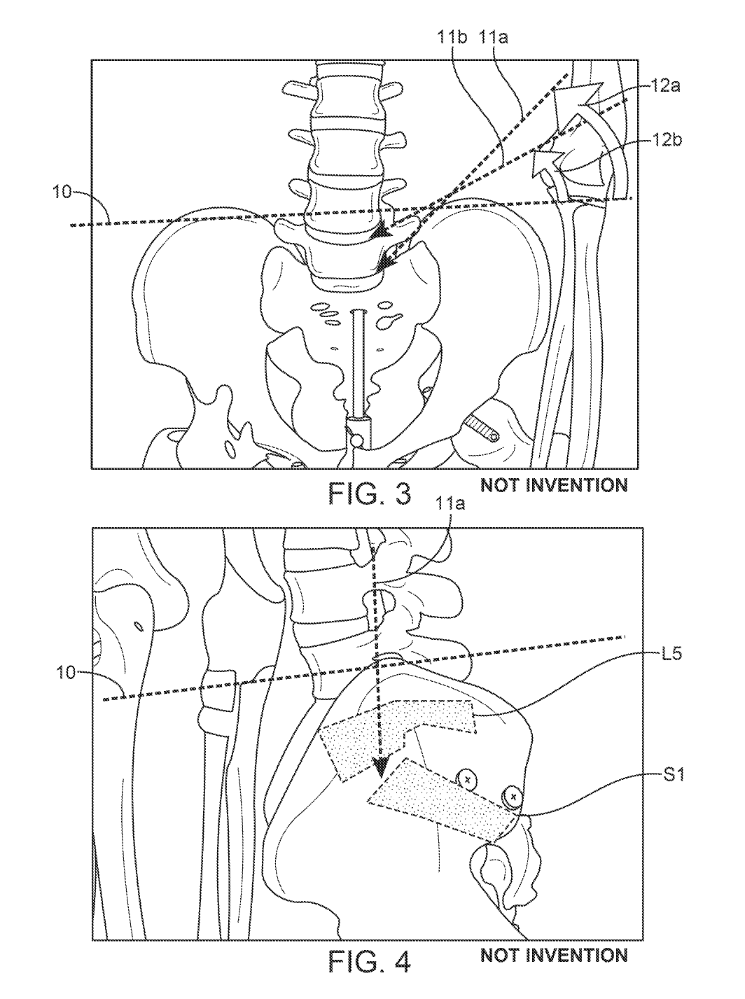

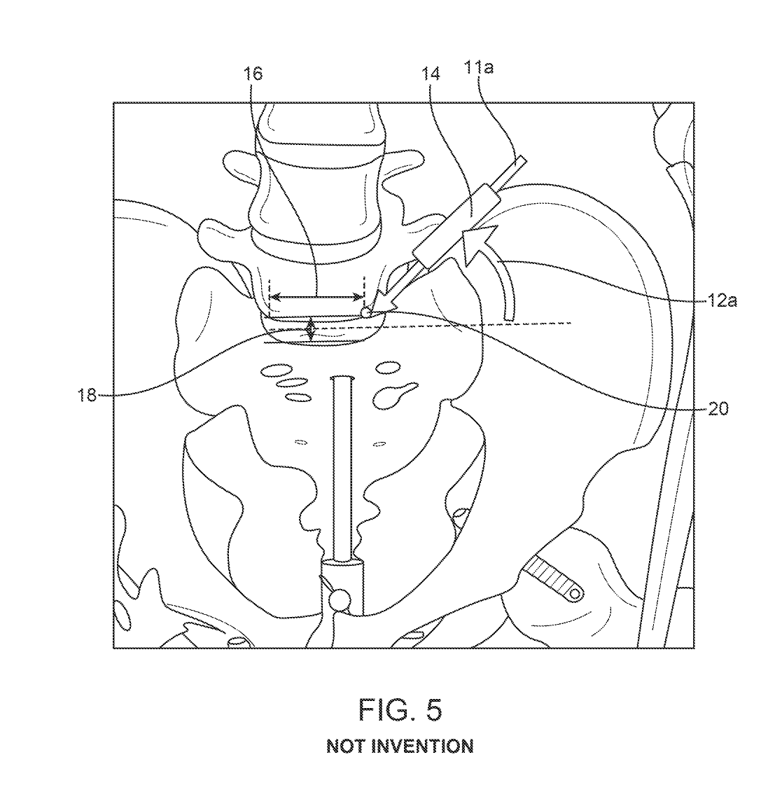

### DETAILED DESCRIPTION

Implantable orthopedic support devices and methods for implanting the same that can access, control (i.e., steer) and deliver the devices into the L5-S1 disc space are disclosed. A tunnel or bone channel 22 can be drilled through the sacroiliac joint. The device 14 can be delivered through the bone channel 22 and into the L5-S1 joint space. The delivery method can be performed without disrupting nerves and major blood vessels. The implant has additional hardware to lock and/or stabilize the implant (e.g., to make an articulatible or flexible implant unarticulatible or rigid) and connect and attach the L5 vertebra to the S1 vertebra.

The support device 14 can be one or more flexible fusion devices, such as cages. The support devices 14 can articulate and/or bend, for example to be able to make a sharp turn from exiting a transosseous bone channel 22 and entering into the L5-S1 disc space. The support devices 14 can have rigid sections 24 connected by articulatable axes 26 (e.g., hinges), or rigid sections 24 and flexible lengths (e.g., lengths integrated with the rigid sections that are made from a more flexible material), or be flexible and/or resilient along the entire length of the device, or combinations thereof.

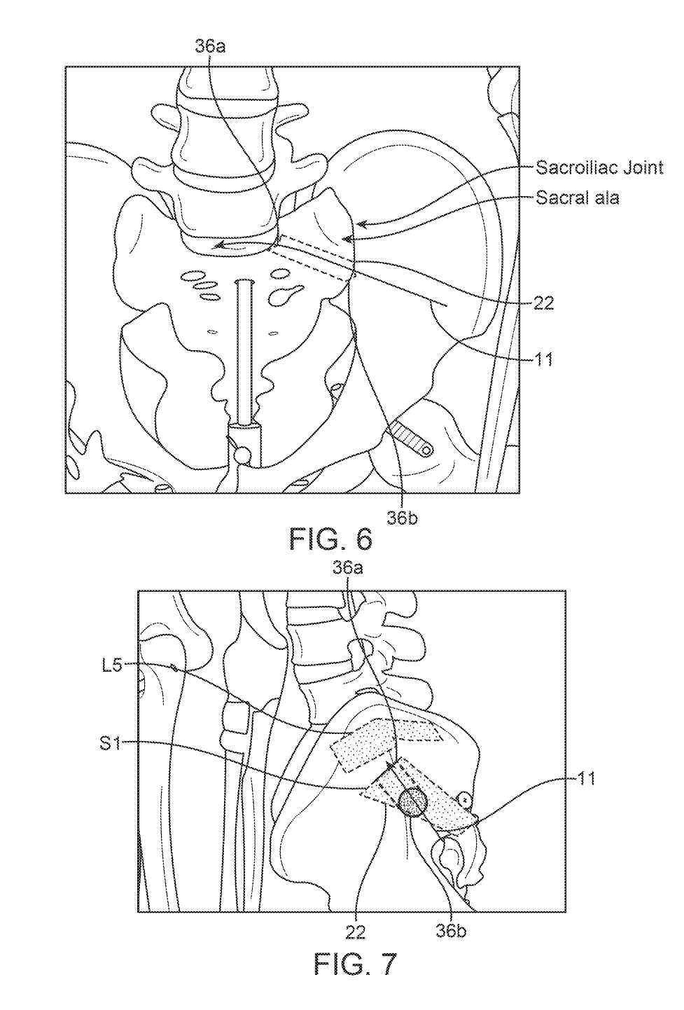

FIGS. 6 and 7 illustrate that the transosseous access bone (i.e., access) channel 22 can be drilled in a position to exit to the L5-S1 intervertebral disc space through the ala of the sacrum. The bone channel 22 can be drilled through the Ilium and/or sacrum. The bone channel 22 can be drilled with a straight and/or flexible or articulatable drill. The bone channel 22 can be hollow.

The bone channel 22 can be fitted with a collar or tube in contact with the perimeter of the channel. The collar or tube can be attached to a trocar. The tube can be delivered into the channel separate from a trocar. The tube can be hollow. The tube can have one, two, three or more lumens. The implant device can be inserted through a lumen in the tube. The tube lumen(s) can have a low friction internal surface. For example, the internal surface of the lumen(s) can be coated with PTFE (e.g., Teflon).

FIG. 7 illustrates a lateral view of the lower spine. The transosseous access bone channel 22 can be oblique and non-perpendicular to the spine. The delivery (i.e., access) path 11 to the L4-L5 or L5-S1 disc space can be oblique and non-perpendicular to the spine. The transosseous delivery path 11 can be through bone (e.g., through the Ilium and sacrum). The delivery path 11 can bypass all or major arteries, veins, muscles, nerves or combinations thereof.

Access tools, such as elongated retractors that can be fit through the superficial incision and/or through the bone channel 22, can move soft tissue out of the way to create access to the channel from the outside of the patient's body. The distal end of the implant device 14 can be atraumatic. For example, the distal terminal end of the device 14 can have a rounded tip to spread or dissect tissue away from the delivery path 11 during translation of the device during delivery.

One or more deployment tools can deliver and deploy the support device 14. The deployment tools can attach to the support device 14 to allow the support device 14 to passively articulate or flex in response to resistive forces from surrounding tissue and/or to actively articulate or flex the support device 14 due to control inputs (e.g., pushing, twisting, button pressing, level manipulation, or combinations thereof) from the user. The interface or connection between the deployment tool and the support device 14 can manipulate the support device 14 by bending, flexing, steering, or combinations thereof. The deployment tool or tools can clear or debride the disk space (i.e., performing a partial or complete discectomy). The deployment tools can articulate and/or flex and follow the delivery paths shown for the support device 14 herein, for example to reach the L4-L5 and/or L5-S1 disc space. The deployment tools can be pre-angled to reach and remove intervertebral disk tissue, for example the deployment tool can be rigid and bent or can flex and articulate.

The support device 14 can fuse adjacent vertebrae to each other. The support device 14 can be used with securing (e.g., nails and screws, for example positioned through the support device 14 and one or both of the adjacent vertebrae) and/or targeting devices (e.g., radiopaque markers).

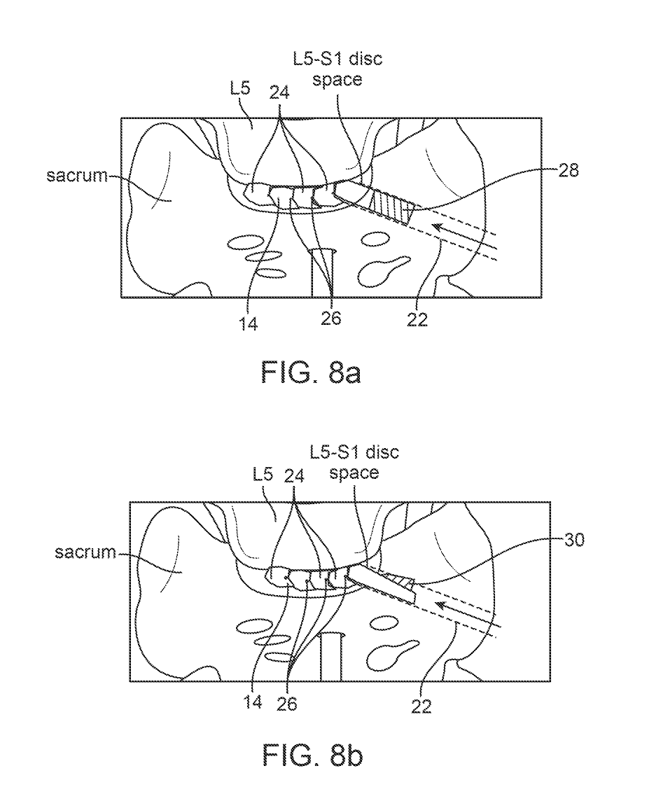

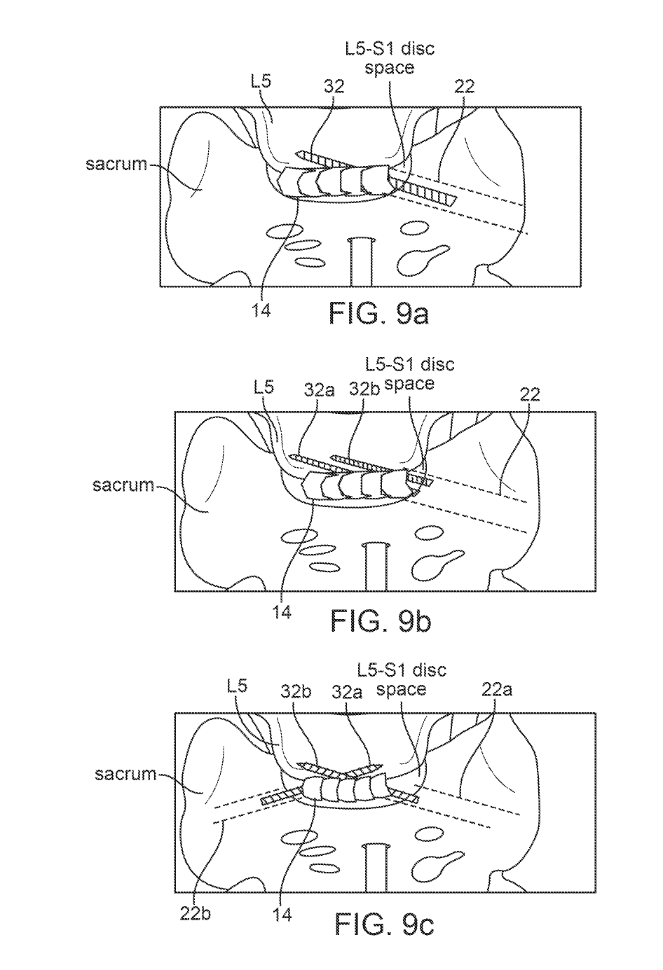

FIGS. 8a through 9c illustrate that additional securing devices and methods can be used to fix, stabilize, help heal, minimize or prevent migration of the support device, reduce bone (e.g., L4, L5, S1, and combinations thereof) movement relative to the support device and relative to the other hones, and combinations thereof. The supplement stabilization elements 28 can secure the position of the flexible implant 14 to the surrounding bone. The support device 14 can completely fuse to the ends plates of the surrounding bone (e.g., L4, L5, S1, and combinations thereof). Other devices for fusing adjacent vertebrae, for example facet fusion elements, pedicle screws and rods, anterior plates, and combinations thereof, can be used in combination with the support device 14.

FIG. 8a illustrates that the support device 14 can be long enough and/or inserted at a length into the disc space so that a portion of the support device 14 can extend into the bone channel 22 after the device 14 is inserted into and oriented in the intervertebral space. The portion of the support device 14 inside of the bone channel 22 can be straight or at an angle to the portion of the support device 14 directly adjacent and on the outside of the bone channel 22. For example, the support device can be flexible through the distal ⅔ of the length of the support device and the proximal ⅓ of the length of the support device can be rigid or not flexible, but articulatable with the distal ⅔ of the length of the support device. The proximal ⅓ of the length of the support device 14 can remain in the sacrum access tunnel or bone channel after the support device 14 is positioned at the target site in the disc space. The stiff proximal section of the support device 14 can be hingedly and/or flexibly connected to the distal length of the support device 14. The support device 14 can be fixed to the bone channel, for example at the proximal length of the support device 14. The proximal end of the support device can be glued, impacted, screwed, or a combination thereof, to the bone channel and/or to a collar in the bone channel.

The proximal and/or distal ends of the support device 14 can have a porous bone ingrowth matrix on the outer surfaces of the support device 14, for example promoting bone growth into the support device 14 fixing the support device to surrounding bone (e.g., in the bone channel 22 and/or L4, L5, and/or S1). The proximal, distal or entire length of the support device 14 can be hollow, cannulated, threaded, have teeth, be expandable, barbed, be multiple pieces, or combinations thereof (e.g., to promote bone growth into the support device). Any or all of the hollow lengths of the support device 14 can be filling with the bone ingrowth matrix before, during or after the device 14 is positioned at the target site.

After the device 14 is positioned at the target site, a screw plug 28 can be inserted, as shown by arrow, through the bone channel 22. The screw plug 28 can have helical threads that can have an outer diameter larger than the diameter of the bone channel 22. The screw plug can be helically rotated through the bone channel 22. The screw plug 28 can fill the bone channel 22. The screw plug 28 can be at the distal or proximal end of the bone channel 22. The screw plug 28 can abut the device 14. The screw plug 22 can be made from PEEK, an allograft, Ti, PE, PMMA, milled bone, steel, any other material disclosed herein, or combinations thereof.

FIG. 8b illustrates that an interference screw 30 can anchor and fix the proximal length of the device 14 to the wall of the bone channel 22. The interference screw 30 can be inserted, as shown by arrow, between the proximal length of the support device 14 and the bone channel 22 and/or between the distal length of the support device 14 and the adjacent bone (e.g., a vertebra). The interference screw 30 can pressure-fit the support device 14 against the bone channel 22 and/or adjacent bone (e.g., a vertebra). The interference screw 30 can be inserted parallel with the longitudinal axis of the length of the support device 14 adjacent to the interference screw 30. The interference screw 30 can have helical threads. The interference screw 30 can have a diameter less than the diameter of the bone channel 22.

FIGS. 9a through 9c illustrate that the support device 14 can have additional transosseous single, double, or crossing lag anchor screws 32, bolts, spears, tacks, other anchors, or combinations thereof, inserted through or around the support device 14 and into surrounding bone and/or soft tissue. The anchor screws 32 can pass through the support device 14 or outside the support device 14 in front, back and/or to the side of the support device 14 (i.e., anterior, posterior and/or laterally).

The outer diameter of the anchor screws 32 can be larger, smaller or the same as the inner diameter of the bone channel 22 or tube lumen inner diameter through which the respective screw is to be delivered. The proximal ends of the anchor screws 32 can be threaded or smooth (e.g., as an anchor pin). The proximal end of the anchor screws 32 can be can be inside a larger diameter plug smaller than, equal to or larger than the bone channel 22 or tube lumen inner diameter. The anchor screws 32 can be rigid.

FIG. 9a illustrates that a single anchor screw 32 can be inserted through a bone channel 22. The bone anchor screw 32 can extend through the device 14 and into an adjacent vertebral body. FIG. 9b illustrates that a first anchor screw 32a and a second anchor screw 32b can be inserted through the bone channel 22. The first anchor screw 32a can be inserted parallel and/or not crossing with second anchor screw 32b. FIG. 9c illustrates that a first anchor screw 32a can be inserted through a first bone channel 22a and a second anchor screw 32b can be inserted through a second bone channel 22b. The first bone channel 22a can be on the same or opposite side of the target site from the second bone channel 22b. The first anchor screw 32a can overlap with the second anchor screw 32b when viewed in the lateral plane.

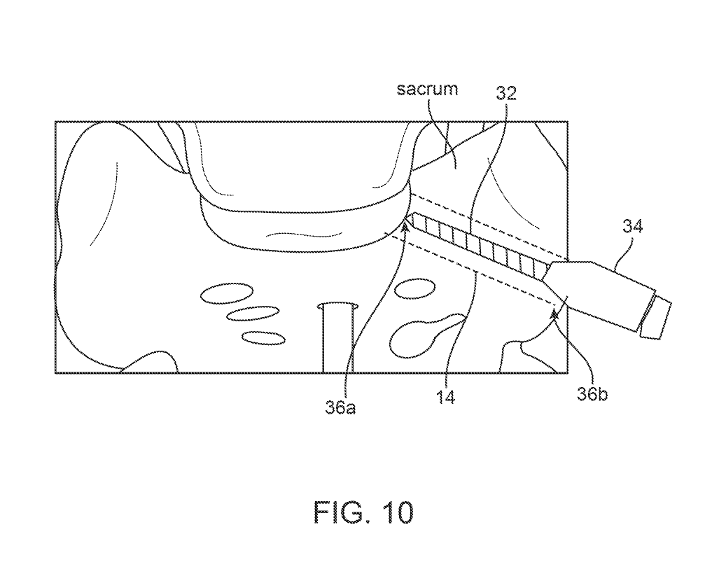

FIG. 10 illustrates that a blocking plug 34 can be inserted into the bone channel and/or tube lumen. The blocking plug 34 can have an outer diameter smaller than, equal to or larger than the inner diameter of the bone channel 22 and/or tube lumen. The blocking plug 34 can taper to a smaller diameter on the distal end of the blocking plug 34. The anchor screw 32 can be fixed to and extend from the blocking plug 34. The blocking plug 34 can be surrounded by bone cement and/or an adhesive. The blocking plug 34 can be used to center the screw. The blocking plug 34 can interference fit with and fill the bone channel 22, for example preventing and/or minimizing migration of the support device 14 and/or anchor screw 32. The blocking plug 34 and/or anchor screw 32 can penetrate or not penetrate the support device 14. The support device 14 can make contact with (e.g., interference fit or abut) the support device 14, for example to hold or brace the support device 14 in a deployed position in the target site. The blocking plug 34 can be inserted to a depth to push on the proximal end of the support device 14 (e.g., to position the support device 14). The blocking plug 34 can be positioned behind the support device 14 (e.g., at the posterior spine), pushing the support device 14 forward (e.g., distally or anteriorly) and blocking the access pathway (e.g., bone channel 22).

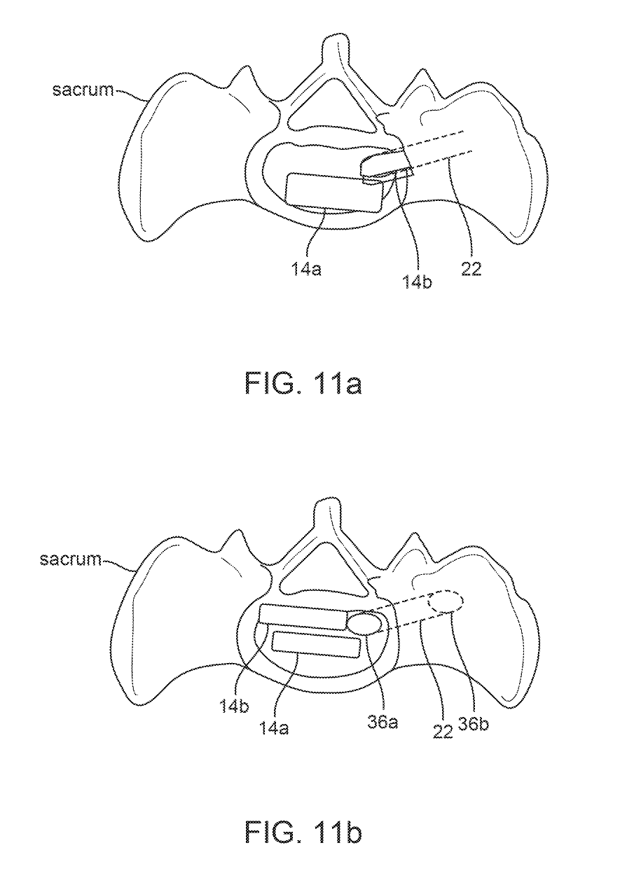

FIGS. 11a and 11b illustrate that a first support device 14a and a second support device 14b can be inserted into the target site. The first support device 14a can be anterior or posterior, lateral or medial, superior or inferior (e.g., in contact or in different disc spaces such as a first support device 14a in the L4-L5 space and the second support device 14b in the L5-S1 space), or a combination thereof of the second support device 14b. For example, identical diameter or different diameter support devices 14 can be inserted through identical diameter or different diameter bone channels 22 and/or tube lumens (e.g., larger diameter support devices 14 can be inserted through larger diameter bone channels and smaller diameter support devices 14 can be inserted through smaller bone channels). The longitudinal axis of the first support device 14a can be positioned parallel with (as shown in FIG. 11b) or non-parallel with (as shown in FIG. 11a) the longitudinal axis of the second support device 14b.

Each bone channel 22 can have a medial bone channel port 36a and lateral bone channel port 36b.

Any or all elements of the device and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET)/polyester (e.g., DACRON® from E. I. Du Pont de Nemours and Company, Wilmington, Del.), polypropylene, (PET), polytetrafluoroethylene (PTFE), expanded PTFE (ePTFE), polyether ketone (PEK), polyether ether ketone (PEEK), poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the device and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

The device and/or elements of the device and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or fillers and/or glues can be osteogenic and osteoinductive growth factors.

Examples of such cements and/or fillers includes bone chips, demineralized bone matrix (DBM), calcium sulfate, corallins hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholerae; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Spl Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

U.S. patent Ser. No. 13/592,271 and PCT Application No. US 12/51945, both filed Aug. 22, 2012, are incorporated by reference herein in their entireties. The broach can be used to perform the discectomy. The elements and characteristics of the broach can be the same as those for the support device 14.

Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

We claim:

1. A biological implant support device for providing orthopedic support comprising:
a first rigid section at a first terminal longitudinal end of the device;
a second rigid section rotatably attached to the first rigid section at a longitudinal end of the first rigid section away from the first terminal longitudinal end of the device; and
wherein second rigid section comprises a thread.

2. The device of claim 1, further comprising an axle rotatably attaching the first rigid section to the second rigid section.

3. A system for providing orthopedic support comprising:
the device of claim 1; and
a first screw attached to and extending away from device.

4. The system of claim 3, further comprising a second screw attached to and extending away from device.

5. A system for providing orthopedic support comprising:
device of claim 1; and
a plug abutting the device.

6. The system of claim 5, wherein the plug comprises a thread.

7. A method for inserting a support device to a target site in a spine adjacent to a first vertebra comprising:
creating a channel through the ala of the sacrum;
inserting a first rigid section of the device through the tunnel and into the target site,
inserting a second rigid section of the device into the tunnel,
rotating the second rigid section of the implant with respect to the first rigid section, wherein the first rigid section is hingedly attached to the second rigid section; and
fixing the second rigid section of the implant in the tunnel.

8. The method of claim 7, wherein creating the channel comprises drilling with a flexible drill.

9. The method of claim 7, wherein the channel has a cylindrical cross-section.

10. The method of claim 7, wherein the non-vertebral bone comprises the pelvis.

11. The method of claim 7, wherein the non-vertebral bone comprises the ilium.

12. The method of claim 7, wherein the non-vertebral bone comprises the sacrum.

13. A method for inserting a support device to a target site in a spine adjacent a first vertebra comprising:
creating a channel through a non-vertebral bone;
inserting the support device through the channel and into the target site; and
filling the channel while the support device is at the target site.

14. The method of claim 13, wherein the channel extends through the pelvis.

15. The method of claim 13, wherein the channel extends through the ilium.

16. The method of claim 13, wherein the channel extends through the sacrum.
